(19) United States
(12) Patent Application Publication
(10) Pub. No.: US 2021/0393291 A1
(43) Pub. Date: Dec. 23, 2021

(54) Transseptal puncture device

(71) Applicant: Murray Rosenbaum, Boca Raton, FL (US)

(72) Inventor: Murray Rosenbaum, Boca Raton, FL (US)

(21) Appl. No.: 17/207,710

(22) Filed: Mar. 21, 2021

Related U.S. Application Data
(60) Provisional application No. 62/963,948, filed on Jan. 21, 2020.

(51) Int. Cl. A61B 17/34; A61B 17/00

(52) U.S. Cl. CPC A61B 17/3462; A61B 2017/00247; A61B 17/00234

(57) ABSTRACT

A transseptal needle for use in a transseptal puncture procedure includes a needle portion connected to a dilating portion. A sheath portion enclosing the needle body includes a relatively compressible transition portion. The transition portion slides back into a receiving portion in the dilating portion during the procedure. The relatively compressible material and shape of the transition portion aids in easing the dilation of the initial puncture in the interarterial septum to accommodate the diameter of the sheath.

### CROSS-REFERENCE TO RELATED APPLICATIONS

This application claims priority to U.S. Provisional Patent Application Ser. No. 62/963,948, filed Jan. 21, 2020, and entitled “TRANSSEPTAL PUNCTURE DEVICE”, the entirety of which is incorporated herein.

### FIELD OF THE INVENTION

The present invention relates to a transseptal puncture device, and more particularly to a transseptal puncture device including a transition portion to ease dilation of the initial puncture at the transition between the dilating portion and the sheath.

### BACKGROUND

Transseptal puncture is a routine procedure for cardiac electrophysiologists to access the left atrium, predominantly for ablation of atrial fibrillation.

The goal of a transseptal puncture procedure is to cross from the right atrium to the left atrium through the Fossa Ovalis, which is located posterior and caudal to the aortic root and anterior to the right atrial free wall. The Fossa Ovalis is approximately 2 cm in diameter and bounded by a ridge of tissue.

A transseptal needle may be used to puncture the Fossa Ovalis. The needle is typically made of a stiff metal cannula with a sharpened distal tip.

As shown in FIG. 1, a transseptal needle 100 is introduced through a guiding sheath in the femoral vein 102 and advanced through the vasculature into the right atrium. From there the needle tip is positioned at the Fossa Ovalis, the preferred location on the interarterial septum for creating a perforation.

Once in position, the user applies force at the proximal end of the needle to advance the needle through the septum and into the left atrium. Once in the left atrium the needle can be attached to an external pressure transducer and the operator can confirm a left atrial pressure before continuing with the procedure.

When using the transseptal catheter to puncture the Fossa Ovalis, a significant amount of force is required to dilate the septum wall to accommodate the thicker portion of the sheath enclosing the needle body after the needle tip has punctured the Fossa Ovalis and been dilated by a dilating portion. Typically there is a ridge between the outer diameter of the dilating portion and the larger outer diameter of the sheath. Gauging the amount of force necessary to sufficiently dilate the septum wall to accommodate the sheath may be difficult, and may vary from patient to patient. If excessive force is used, the needle point may overextend into the left atrium and cause damage.

Accordingly, it would be advantageous to provide a transseptal puncture device that allows for an easier transition, i.e., less force, while dilating the septum wall into the left atrium to accommodate the diameter of the sheath.

### SUMMARY

Aspects of the present invention provide a transseptal needle for use in a transseptal puncture procedure may includes a needle portion which may be connected to a dilating portion. A sheath portion enclosing the needle body includes a transition portion made from a relatively compressible material, e.g., silicone, compared to the more rigid materials used for the needle portion, dilating portion, and sheath portion.

The transition portion slides back into a receiving portion in the dilating portion during the procedure. The relatively compressible material and shape of the transition portion aids in easing the dilation of the initial puncture in the interarterial septum. For example, the transition portion may have a wedge shape that provides a transition between the outer diameter of the dilating portion and the outer diameter of the sheath, providing a relatively easy transition when moving the sheath through the tissue wall.

During the procedure, the relatively compressible material of the transition portion may ease dilation of the initial puncture by the needle portion and dilating portion to the outer diameter of the sheath.

A method for using the transseptal needle according to an embodiment includes puncturing the Fossa Ovalis. After puncturing the Fossa Ovalis, the dilating portion is moved through the Fossa Ovalis and then the interarterial septum is punctured. Force is then applied to push the relatively compressible transition portion into a fully engaged position in the receiving portion of the transseptal needle. Force is then applied to push the transition portion through the interarterial septum.

### BRIEF DESCRIPTION OF THE DRAWINGS

The accompanying drawings, which are incorporated herein and constitute part of this specification, illustrate exemplary aspects of the claims, and together with the general description given above and the detailed description given below, serve to explain the features of the claims.

FIG. 1 shows the positioning of a transseptal puncture procedure in a human body.

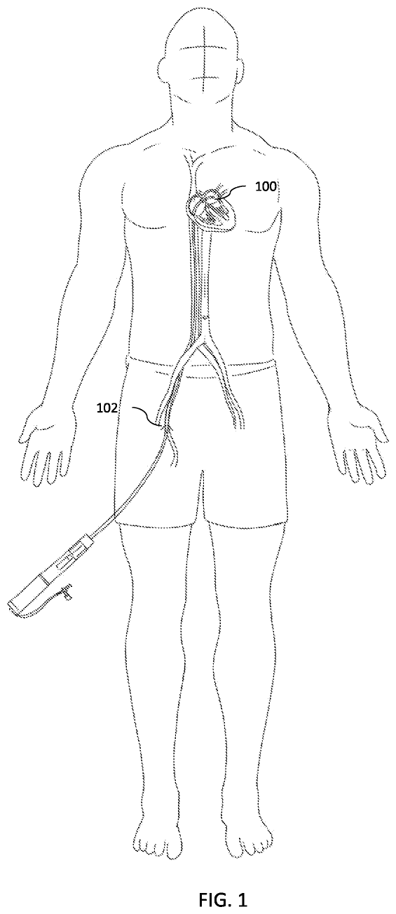

FIG. 2 shows a transseptal needle with the needle point in position to puncture the Fossa Ovalis.

FIG. 3 shows the transseptal needle after puncturing the Fossa Ovalis.

FIG. 4 shows the transseptal needle after puncturing the interarterial septum.

FIG. 5 shows a dilating portion of the transseptal needle provided to ease entry of the needle body through the septum.

FIG. 6 shows transseptal needle according to an embodiment that includes a relatively rigid dilating portion and a relatively compressible transition portion near the needle point.

FIG. 7 shows a bottom edge of the transition portion entering a receiving portion in the dilating portion

FIG. 8 shows another transitional position of the transition portion moving into the receiving portion.

FIG. 9. shows the transition portion fully engaged in the receiving portion.

FIG. 10 is a flowchart describing a method for using a transseptal needle according to an embodiment.

### DETAILED DESCRIPTION

Various aspects will be described in detail with reference to the accompanying drawings. Wherever possible, the same reference numbers will be used throughout the drawings to refer to the same or like parts. References made to particular examples and implementations are for illustrative purposes, and are not intended to limit the scope of the claims.

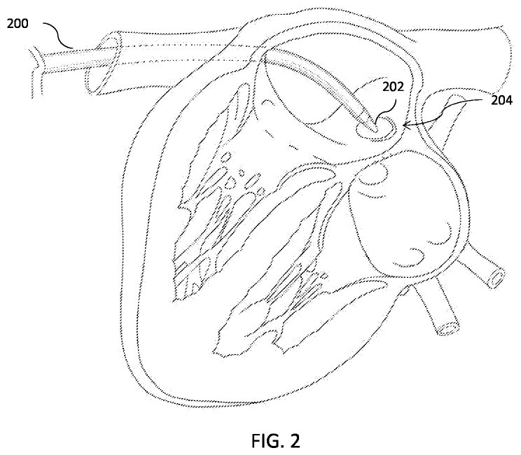

FIG. 2 shows a step in a transseptal puncture procedure in which a transseptal needle 200 including a needle point 202, which is in position to puncture the Fossa Ovalis 204. The needle may have a sharp point, or alternatively a blunt tip needle which uses a burn to penetrate the tissue wall.

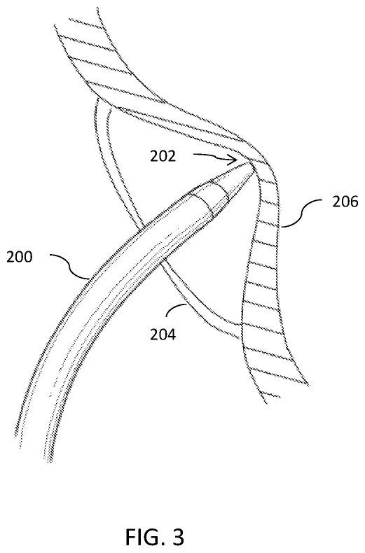

FIG. 3 shows the transseptal needle 200 after puncturing the Fossa Ovalis 204, and positioned to puncture the interarterial septum 206 prior to entering the left atrium. At this point, the dilator may be slid over the needle slightly, and the needle then removed and replaced by a guide wire to be placed in one of the pulmonary veins.

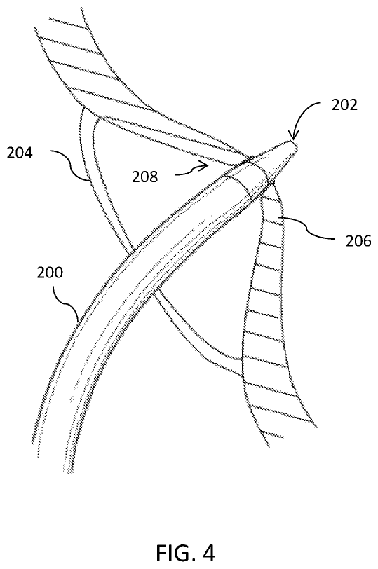

Typically there is a ridge between the outer diameter of the dilating portion and the larger outer diameter of the sheath. After puncturing the interarterial septum 206, as shown in FIG. 4, the initial puncture must be dilated to accommodate the larger diameter of the sheath of the transseptal needle 200. Typically, this portion of the procedure requires a significant amount of force to be exerted by the user to fully penetrate the septum. If excessive force is used, the needle point 202 may overextend into the left atrium and cause damage.

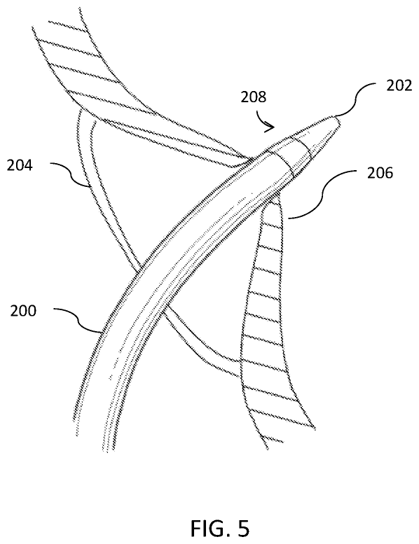

In an embodiment, a dilating portion 208 may be provided to ease entry of the needle body through the septum, as shown in FIG. 5.

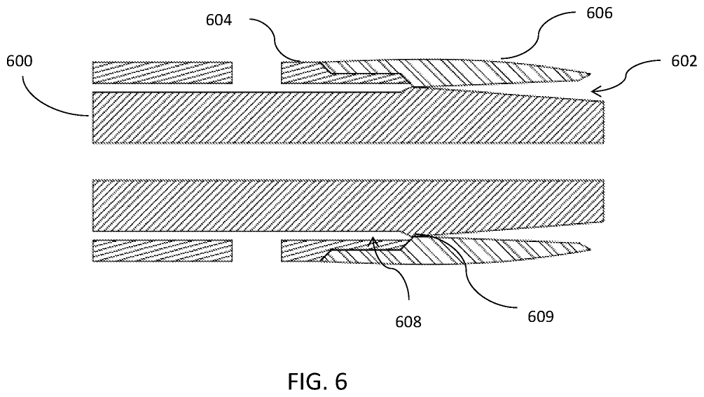

FIG. 6 shows an embodiment of a transseptal needle 600 that includes a relatively rigid dilating portion 602 near the needle point (not shown). A sheath 604 made of a rigid or semi-rigid material encases the remainder of the transseptal needle body. As described above, in conventional transseptal needle designs, the sheath portion typically has a greater diameter than the end of the dilating portion. This forms a ridge which can impede further entry of the needle body into the Fossa Ovalis, and may require a significant amount of force to overcome.

According to an embodiment, a transition portion 606 connected to the sheath 604 may be mounted over the dilating portion. The transition portion 606 may be, e. g., silicone, or other material more compressible and/or flexible than the materials of the dilating portion 602 and sheath 604.

As the needle portion punctures the Fossa Ovalis and/or the septum, and the dilating portion 602 traverses through, the transition portion 606 is pushed back on the body of the dilating portion, ultimately into a ridged receiving portion 608 in the body of the dilating portion. The ridged receiving portion 608 has a smaller outer diameter than the largest diameter 609 of the dilating portion 604.

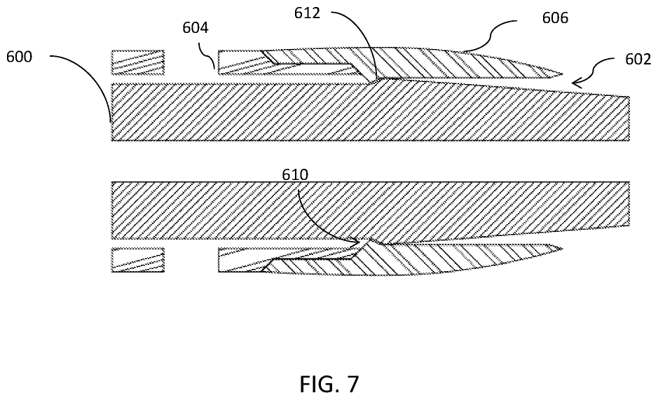

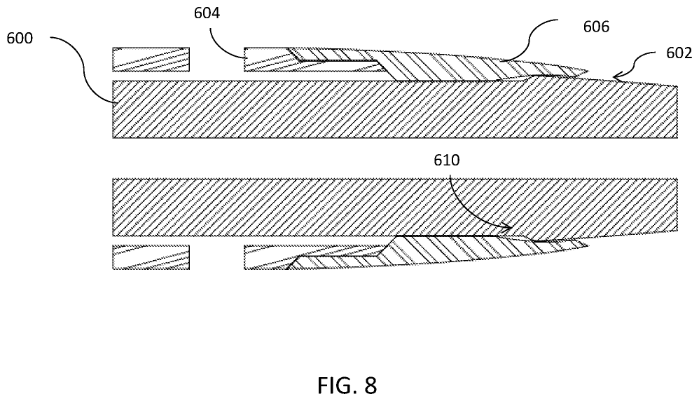

FIG. 7 shows a bottom edge 610 of the transition portion 604 entering the receiving portion 608. As shown, the compressible nature of the transition portion 606 allows it to conform to the shape of the receiving portion, for example, at portion 612. FIG. 8 shows another transitional position of the transition portion moving into the receiving portion 608 as it is pushed back by the tissue of the Fossa Ovalis and/or septum.

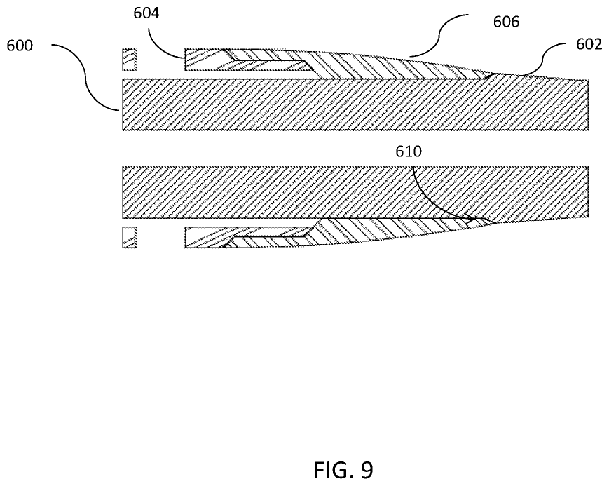

FIG. 9. shows the transition portion 606 fully engaged, or locked, in the receiving portion 608. In the engaged position, the compressible material of the transition portion 606 forms a smooth transition between the diameter of the end of the dilating portion 602 and the diameter of the sheath 604. The compressible nature of the transition portion 606 further assists in easing the sheath and remainder of the transseptal needle through the septum.

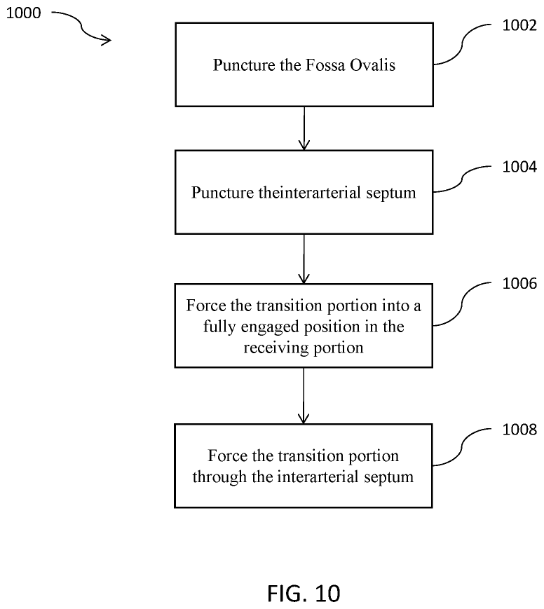

FIG. 10 is a flowchart describing a method 1000 for using a transseptal needle according to an embodiment. The transseptal needle is used to puncturing the Fossa Ovalis (block 1002). After puncturing the Fossa Ovalis, the dilating portion 602 is moved through the Fossa Ovalis and then the interarterial septum is punctured (block 1004). Force is then applied to push the relatively compressible transition portion into a fully engaged position in the receiving portion of the transseptal needle (block 1006). Force is then applied to push the transition portion through the interarterial septum (block 1008).

The foregoing method descriptions and figures are provided merely as illustrative examples and are not intended to require or imply that the operations of various aspects must be performed in the order presented. As will be appreciated by one of skill in the art, the order of operations in the aspects described herein may be performed in any order. Words such as “thereafter,” “then,” “next,” etc. are not intended to limit the order of the operations; such words are used to guide the reader through the description of the methods and systems described herein. Further, any reference to claim elements in the singular, for example, using the articles “a,” “an,” or “the” is not to be construed as limiting the element to the singular.

The principal features of this invention may be employed in various embodiments without departing from the scope of the invention. Those of ordinary skill in the art will recognize numerous equivalents to the specific procedures described herein. The specific embodiments discussed herein are merely illustrative of specific ways to make and use the invention and do not delimit the scope of the invention.

1. A transseptal puncture device comprising:
a body;
a dilating portion adapted to accept a needle; and
a sheath portion slideably mounted over the body, the sheath portion including a transition portion on a first end of the sheath portion,
wherein the dilating portion includes a recessed receiving portion to receive the transition portion when slid into a locked position.

2. The transseptal puncture device of claim 1, wherein the transition portion comprising a material relatively compressible with regard to the sheath portion and the dilating portion.

3. The transseptal puncture device of claim 2, wherein the material of the transition portion comprises silicone.

4. The transseptal puncture device of claim 1, wherein an end of the dilating portion has a first diameter, and
wherein the sheath portion has a second diameter, the second diameter being greater than the first diameter, and
wherein the transition portion has a wedge shape including a surface providing a smooth transition between the first diameter and the second diameter.

5. A method for using a transseptal needle including a slideable sheath including a compressible transition portion, a receiving portion, and a dilating portion, the method comprising:
puncturing the Fossa Ovalis of a patient;
puncturing the interarterial septum of the patient;
applying force to push the compressible transition portion into a fully engaged position in the receiving portion adjacent an end of the dilating portion of the transseptal needle; and
applying force to push the transition portion through the interarterial septum.
